(19) United States
(12) Patent Application Publication
(10) Pub. No.: US 2008/0003278 A1
(43) Pub. Date: Jan. 3, 2008

(54) Food products and dietary supplements for improving mental performance

(76) Inventor: Fernando Calvo Mondelo, Madrid (ES)

Correspondence Address: WILLIAM COLLARD;COLLARD & ROE, P.C., 1077 NORTHERN BOULEVARD, ROSLYN, NY, 11576, US

(21) Appl. No.: 11/476,544

(22) Filed: Jun. 28, 2006

(51) Int. Cl. A61K 36/63; A61K 31/4745

(52) U.S. Cl. 424/451; 424/725; 424/769; 514/283

(57) ABSTRACT

Formulas for producing compositions for the dietary supplement and nutraceutical products, containing virgin olive oil, vitamins, substances as vincamine, vinpocetine or vinburnine, as well as herbal extracts from Vinca Minor and Crioceras longiflorus. The present invention also relates to the oral administration of these compounds, to alleviate mental fatigue or poor cognitive function and anti-ageing prevention.

### BACKGROUND OF THE INVENTION

1. Field of the Invention

The present invention is in the field of dietary supplements and functional foods. More particularly, the present invention relates to dietary supplements and functional food products, containing compositions for the structural/functional nutritional support for those who struggle with poor focus, concentration and/or memory. and anti-ageing. In addition, the present invention provides compositions comprising nutritional/botanical factors helpful to those who subjectively experience transient mental fatigue, or poor cognitive function and ageing symptoms.

2. Brief Description of Background Art

The role of nutrition and the positive influence of dietary-nutritional, herbal-botanical ingredients as they relate to optimal energy production, neurophysiology, and neurotransmitter synthesis/formation cannot be understated.

Physiology textbooks describe the brain as the most metabolically demanding of all organs. Representing only 2% of the total body weight, the brain consumes 50% of the circulating blood glucose, and over 20% of circulating oxygen. Neurons essentially have twice the energy requirements as those of other cells. Since neural requirements for energy substantially exceed those of other cells in the body, Krebs-Cycle intermediates are metabolically essential ingredients, for optimal ATP generation, optimal neural metabolism, and thus, improved mental acuity.

Also, neurotransmitters are naturally occurring molecules that act as biochemical messengers, relaying nerve signals between neurons. Adequate production of the different types of neurotransmitters is responsible for proper mental functioning. Deficiencies of these neurotransmitters interface with behaviour, mood, concentration and memory.

Based on the brain's need for energy production and neurotransmitter synthesis, scientists postulate that the chemistry of our diet is a critical element in the subsequent triggering of neurotransmitter synthesis, and efficient energy production, which jointly lead to normal/optimal cognitive function.

Thus, a new class of research has evolved, that investigates the effect of various dietary, nutritional and herbal constituents, known to improve learning and memory. This type of “smart nutrients and foods” has been termed nootropics meaning literally “toward the mind”.

Exhaustive analytical investigation into nootropics has been ongoing, and several studies have confirmed the necessity of several key nutritional ingredients for mental health. In particular, researchers at the USDA (United States Department of Agriculture) carried out a study to examine how marginal nutritional deficiencies affect memory and mental function. They meticulously determined the nutritional status of twenty-eight healthy people aged sixty and above, and then gave them challenging mental tasks to measure cognitive function.

Significant relationships were noted between nutritional status and test performance. Subjects who had optimal levels of certain nutrients tested better than those with nutrient deficiencies.

Previous studies have demonstrated that vitamins like B2 (riboflavin) act as coenzyme on the metabolism of carbohydrates, lipids and proteins. It is essential for cellular energy needs.

Other types were also predictive of performance on mental function tests. A long term study performed at the University of New Mexico School of Medicine followed up on 137 people, aged between sixty-six and ninety, for six years. In the concluding stage of the study they were given tests to determine cognitive functions. Test performance was related to past and current nutritional status, and significant associations between mental function and vitamin status were noted.

The participants in this study who, on their own, had taken vitamin supplements, did better on difficult visual and spatial tests and on tests of abstract thinking.

Epidemiological studies suggest that a Mediterranean diet decreases the risk of cardiovascular disease.

The Mediterranean diet, rich in virgin olive oil, improves the major risk of cardiovascular disease, such as the lipoprotein profile, blood pressure, glucose metabolism and antithrombotic profile.

Endothelial function, inflammation and oxidative stress are also positively modulated. Some of these effects are attributed to minor components of virgin olive oil. Microconstuents from virgin olive oil are bioavailable in humans and have shown antioxidant properties and capacity to improve endothelial function.

Furthermore, they are also able to modify the haemostasis, showing antithrombotic properties. The more recent studies consistently support that the Mediterranean diet, based on virgin olive oil, is compatible with healthier ageing and increased longevity.

Vitamin E is a major lipid-soluble antioxidant, and is the most effective chainbreaking antioxidant within the cell membrane where it protects membrane fatty acids from lipid peroxidation.

This invention provides unique formulas of nootropic nutrients of natural origin and herbal extracts combined with vitamins and virgin olive oil designed to provide specific dietary-nutritional support factors for cognitive function. Oral administration of the compositions based on these formula results in the efficient formation of mental energy and the synthesis of key neurotransmitters associated with memory, focus and concentration.

The present formulas have increased bioavailability of constituent components and therefore have an enhanced synergistic effect on mental health.

As a convenient addition to the daily diet, the formulas of the present invention provide a unique combination of energy precursors. neurological support antioxidants and nutrients, as well as many nootropic ingredients in their most bioavailable/absorbable forms to provide enhanced efficacy. The extracts of Vinca minor and Crioceras longiflorus should not be mixed with Gingko biloba or other similar extracts because they could cause interactions and adverse effects. The available data do not contain information to support these extracts or about their added effects and safe doses. In any case the doses of both compounds have to be diminished to avoid an exaggerated platelet aggregation increase.

### SUMMARY OF THE INVENTION

The present invention provides compositions, formulated to provide support for anti-ageing and mental performance and/or improve mental performance, through the elimination of mental fatigue, as well as to improve memory, focus and concentration. The present invention provides novel compositions, containing vitamins, virgin olive oil and specific dietary-nutritional products of vegetal origin, like Vinca Minor or Crioceras longiflorus extracts or vincamine, vinburnine and vinpocetine isolated from natural sources. These compositions provide a combination of specific cognitive support factors.

Although individually many of the nutrients have been clinically shown to enhance mental energy levels as well as support and enhance mental focus, concentration and memory, these nutrients have an enhanced synergistic effect when combined within the present composition.

The present invention constitutes novel, proprietary formulas to provide specific dietary-nutritional and herbal-botanical support factors for cognitive function, and antioxidant and anti-ageing effects.

### DETAILED DESCRIPTION OF THE INVENTION

The following specification sets forth the preferred embodiments of the present invention. The embodiments of the invention disclosed herein are the best modes contemplated by the inventors for carrying out their invention in a commercial environment, although it should be understood that various modifications can be accomplished within the parameters of the present invention.

The present invention provides a series of simple nutritional supplements to improve and maintain memory, and increase longevity.

Components of the composition: The composition of this invention consists fundamentally of the following ingredients: virgin olive oil, vitamin B2 (riboflavin), vitamin E (tocopherol) and extracts of Vinca minor, or crioceras longiflorus, or vincamine (natural origin), or vinburnine (natural origin), or vinpocetine (natural origin) in soft gelatine capsules:

Vitamins: vitamin B2 (riboflavin) acts as coenzyme on metabolism of carbohydrates, lipids and proteins. It is essential for cellular energy needs. Vitamin E (tocopherol) is a fat-soluble antioxidant that plays numerous protectant and physiological roles in the cellular metabolism and is the most effective chain-breaking antioxidant within the cell membrane where it protects membrane fatty acids from lipid peroxidation. The compositions contain preferably from 5 mg to 7 mg of those vitamins.

Virgin olive oil: is the principal (often exclusive) and most typical source of visible fat of the Mediterranean diet. The healthful properties of olive oil have been often attributed to its high monounsaturated fatty acid (MFA) content, namely in the form of oleic acid (18:ln-9). However, olive oil poliphenols at nutritionally relevant concentrations transcriptional inhibit endothelial adhesion molecule expression. The latest studies strongly suggest that dietary polyphenols can stimulate antioxidant transcription and detoxification defence systems through antioxidant responsible elements.

These compositions contain preferably from about 140 mg to 160 mg of olive oil.

Vinca Minor extract: This is the common periwinkle, a herbaceous plant with trailing stems that occasionally root into the ground, bearing persistent, opposite, and tough leaves with shiny blades. It is used for circulatory disorders, cerebral circulatory impairment and support for the metabolism of the brain. It is also used for loss of memory, hypertension, cystitis, gastritis and enteritis, diarrhoea, high blood sugar levels and to help weaning. The compositions contain preferably from about 18 mg to 22 mg of Vinca minor extract.

Crioceras longiflorus: It is a small bush or tree, belonging to the apocynaceae plant family. It grows in Africa, more specifically in the Central African region; the C. longiflorus is a plant whose alkaloids opened a vast therapeutic field. The major alkaloid is the 17,18-dehydrovincamine and its epimer 16-epi-17,18 dehydrovincamine. When these extracts are hydrogenated they give an extract that contains vincamine. The compositions contain preferably from about 5 mg to 10 mg of Crioceras longiflourus extract.

Vincamine: is the major alkaloid present in the Vinca Minor extract. Its use is recommended as a cerebral vasodilator in neo-natal calves, for cerebral anoxia. It is widely used in human medicine, to increase global and regional blood flow in patients suffering from acute or sub chronic cerebral ischemia. It occurs naturally in Vinca Minor leaves. The composition contains preferably from about 5 mg to 10 mg of vincamine.

Vinburnine: is an alkaloid related to vinca and other related species. activator of cerebral metabolism by enhancing blood flow. It has been studied in the treatment of cerebral insufficiency with cognitive dysfunction. It occurs naturally in the Hunteria Eburnea plant. The composition contains preferably from about 5 mg to 10 mg of vinburnine.

Vinpocetine: is a nootropic derived from plants. It has been shown to improve blood flow, circulation and oxygen utilization in the brain of animals and humans. It also protects neurons from the devastating effects of disrupted oxygen delivery. It is, therefore, a useful therapy for symptoms of senile dementia and cerebral vascular insufficiency.

Vinpocetine also improves energy production in brain cells. It has been studied as a memory booster for young, healthy people, in whom it has been shown to improve short-term memory. In addition, it appears to have anticonvulsant properties. A Russian study of epileptic patients demonstrated that vinpocetine reduced the frequency and, in some cases, completely eliminated epileptic seizures in twenty of the thirty-one patients participating in the study. It occurs naturally in Crioceras Longiflorus root bark. The compositions contain preferably from about 1 mg to 2 mg of vinpocetine.

Formulation 1: A soft capsule contains 3 mg of vitamin E, 3 mg of vitamin B2, 150 mg of virgin olive oil, 20 mg of vinca minor extract and excipients.

Formulation 2: A soft capsule contains 3 mg of vitamin E, 3 mg of vitamin B2, 150 mg of virgin olive oil, 10 mg of crioceras longiflorus (hydrogenated extract) and excipients.

Formulation 3: A soft capsule contains 3 mg of vitamin E, 3 mg of vitamin B2, 150 mg of virgin olive oil, 10 mg of vincamine and excipients.

Formulation 4: A soft capsule contains 3 mg of vitamin E, 3 mg of vitamin B2, 150 mg of virgin olive oil, 10 mg of vinburnine and excipients.

Formulation 5: A soft capsule contains 3 mg of vitamin E, 3 mg of vitamin B2, 150 mg of virgin olive oil, 2 mg of vinpocetine and excipients.

### BIBLIOGRAPHY

1.—Smith Kyl L, USP 20050244510, November 2005.
2.—Gilbert Gluk et al.: U.S. Pat. No. 6,228,418, May 2003.
3.—Guyton, A. C. Textbook of Medical Physiology, 10th Edition, WB. Saunders Co 2000.
4.—Tucker, D. M. et al. “Nutrition status and brain function in aging”. American Journal of Clinical Nutrition, 52 (1): 92-102, July 1990.
5.—La Rue, A. et al. “Nutritional status and cognitive functioning in a normal aging sample: a 6-year reassessment”. American Journal of Clinical Nutrition, 65 (1): 20-29, January 1997.
6.—Perez-Jimenez, F.; “International conference on the healthy effect of virgin olive oil”; Eur. J. Clin. Invest., 35 (7): 421-424, July 2005.
7.—Visioli, F. et al.; “Mediterranean food and health: building human evidence’: J. of Physiology and Pharmacology, Suppl. 1, 37-49. 2005.
8.—Carluccio, M. A., et al.:. “Olive oil and red wine antioxidant polyphenols inhibit endothelial activation: antiatherogenic properties of Mediterranean diet phytochemicals”, Arteriosclerosis, Thrombosis, and Vascular Biology, J. of the American Heart Ass., 23: 622-629, February 2003.
9.—De la Puerta, R., and Ruiz-Gutierrez, V., “Effect of minor constituents of virgin olive oil on topical antiinflammatory assays”; Zeitschrift fur Naturtorschung C-A Journal of Biosciences; 55 (9/19) 814-819, 2000.
10.—De la Puerta. R., and Ruiz-Gutierrez, V.'. “Efecto protector de los polifenoles del aceite de oliva virgen en sistemas de liberación de radicales libres y eicosanoles”, Clínica e Investigación en Arteriosclerosis, 12 (4). 183190;2000
11.—Masella, R., et al.: “Novel mechanism of natural antioxidant compounds in biological systems: involvement of glutathione and glutathione-related enzymes”; J-Nutr. Biochem., 16 (10): 577-586, 2005.
12.—“The Vitamine E”. French Wikipedia, July 2004.
13.—Ravindra, P. S., et al., “Free radicals and oxidative stress in neurodegenerative diseases: relevance of dietary antioxidants”; JIACM, 5(3): 218-25 2005.
14.—“Estres oxidativo: la paradoja del oxígeno”, Rev. Cubana Endocrinol. 11(3): 139-142:2000.
15.—Hart S.; “Coronary Herat disease and antioxidant vitamin E”, Nutrition bytes, 3 (1), article 2, 1997.
16.—‘Vitamina B2: riboflavina’, Goodman and Gilman.
17.—Okuyama S. wt al. “Effects of VA-045, a novel apovincaminic acid derivative on age-related imparement evidence in electroencephalograph, caudate spindle, a passive avoidance task and cerebral blood flow in rats” General Pharmacology, 25 (/): 1311-1320, 1994.
18.—Molnar P., Erdo S. L.; “Vinpocetine is as potent as phenytoin to block voltage gated Na+ channels in rat cortical neurons”, Eu. J. of Pharmacology, 273: 303306.1995.
19.—Paulo T. et al.; “[3H] Noradrenaline-releasing action of vinpocetine in the isolated mail pulmonary artery of the rabbit”, J. Pharm. Phramacol, 38: 668-673, 1986
20.—Oyomo E. et al.; “Comparison of vinpocetine with ifenprodil tartrate and dihydroergotoxine mesylate treatment and results of long-term treatment with vinpocetine”; Current Therapeutic Research, Vol 37 (5): 811-821, 1985.
21.—Manconi E. et al., “A double-blind clinical trial of vinpocetine in the treatment of cerebral insufficiency of vascular and degenerative origin”: Current Therapeutic Research, Vol 40 (4): 6702-709, 1986.
22.—Balestreri R. et al. “A double-blind clinical trial of the safety and efficacy of vinpocetine in the treatment of patients with chronic vascular senile cerebral dysfunction”: J. of Am. Geriatrics Soc.; 35: 425-430, 1987.
23.—Mizazaki M.; “The effect of a cerebral vasodilator, vinpocetine, on cerebral vascular resistance evaluated by the dopplar ultrasonic technique in patients with cerebrovascular diseases”. Angiology, The Journal of Vascular Diseases, 1 (46): 53-58,1995.
24.—Tretter L. and Adam-Vizi V.; “The neuroprotective drug vinpocetine prevents vertridine-induced [Na+] and [Ca++] rise in synaptosomes”, NeuroReport, 9: 1849-1853,1998.
25.—Cavé A. “Pharmacognosy, Phytochemistry, Medicinal Plants” Ed. Lavoisier. pags.: 838-840.
26.—FDR for Herbal Medicines, pags.: 884-885.
27.—“Periwinkle”; The Commission E Monographs.
28.—Food for Thought—Herbal Brain Boosters.
29.—Vachnadze V. Yu et al,‘ “Chemical composition and pharmacological activity of alkaloids from the common periwinkle cultured in Georgia”; Pharmaceutical Chemistry Journal, 35 (5): 268-270, May 2001.
30.—Cavé A. et al.; “Alcaloides du C. longiflorus”, C.R. Acad. Sc. Paris, 272: 2367-1369: abril 1971.
31.—Olpe H. R. et al.; Life Sci. 31(18): 1947-1953, 1982.
32.—Fischhof P. K. et al.; Neuropsychobiology, 34(1): 29-35. 1996.
33.—Machova J. Et al.; Eur. J. Phramacol. 162 (3): 387-395, 1989.
34.—Ritschel W. A. et al., Res Común Chem Pathol. Pharmacol 48(2): 221-42, 1985.
35.—Rassat et al.; Naunyn Schmiedebergs Arch Pharmacol. 318 (4): 349-357, 1982.
36.—Millart H. Et al., Int. J. Clin. Pharmacol. Ther. Toxicol. 21 (11). 581-586. 1983.
37.—Ritschel W A, Agrawala P.; Methods Find Exp. Clin. Pharmacol. 7(3): 129136,1985.
38.—EMEA/MRL/587/99-FINAL, April 1999.
39.—Geffen D.; “The effects of Gingko biloba on learning and memory”; Nutrition Noteworthy, 3 (1): article 1, 2000.
40.—Vas A et al., “Eburnamenine derivatives and the brain”, Med. Res. Rev., 25(6):737-757,2005.
41.—Louajri A. et al.; “Red blood cell metabolism and haemoglobin oxygen affinity. Effect of Vinburnine on normobaric hypoxic rats”, Biol Pharm Bull., 22(8):773-774,1999.
42.—Spadaro F. et al.; “Effects of vinburnine on experimental models of learning and memory impairments”, Pharmacol Biochem Behav, 37(1):53-57, September 1990.
43.—Domini L. et al.; “The effects of the acute administration of vinburnine on blood rheology, oxygen transport and regional circulation under normal conditions and in hypoxemia. In vivo research”; Recenti Prog Med, 81(11): 716723,1990.

What is claimed is:

1. An soft capsule for anti-ageing and enhanced mental function, which comprises:
a. Vitamins E and B2;
b. Virgin olive oyl; and
c. Vinca minor extracts or Crioceras longiforous extracts or vincamine or vinburnie or vinpocetine.

2. The soft capsule according to claim 1 wherein the vitamins E and B2 are within the range from 140 mg to 7 mg. olive oil and 20 mg of Vinca minor extract

3. The soft capsule according to claim 1, wherein virgin olive oil is within the range from 140 mg to 160 mg.

4. The soft cpasule according to claim 1, wherein Vinca minor extracts are within the range from 18 mg to 22 mg.

5. The soft capsule according to claim 1, wherein Criocera longflorus extracts, vincamine or vinburnine are within the range from 5 mg to 10 mg.

6. The soft capsule according to claim 1, wherein vinpocetine is within the range from 1 mg to 2 mg.

7. (canceled)

8. The soft capsule according to claim 1, which contains 3 mg of vitamine E, 3 mg of vitamin B2, 150 mg of virgin olive oil and 20 mg of Vinca minor extract.

9. The soft cpasule according to claim 1, which contains 3 mg of viatmin E, 3 mg of viatamin B2, 150 mg of virgin olive oil and 10 mg of Crioceras longiflorus extract (hydrogenated extract).

10. The soft capsule according to claim 1 which contains 3 mg of vitamin E, 3 mg of vitamin B2, 150 mg of virgin olive oil and 10 mg of vincamine.

11. The soft capsule according to claim 1 which contains 3 mg of vitamin E, 3 mg of vitamin B2, 150 mg of virgin olive oil and 2 mg of vinburnine.

12. The soft capsule according to claim 1 which contains 3 mg of vitamin E, 3 mg of vitamin B2, 150 mg of virgin olive oil and 2 mg of vinpocetine.
